(19) United States
(12) Patent Application Publication
(10) Pub. No.: US 2021/0145487 A1
(43) Pub. Date: May 20, 2021

(54) MEDICAL IMPLANT HAVING A MULTI-MODE LOCKING MECHANISM AND METHOD OF USE THEREOF

(71) Applicant: ALLIANCE PARTNERS LLC, San Antonio, TX (US)

(72) Inventors: Karl W. Swanhn, San Antonio, TX (US); Gregory Andrew Grim, San Antonio, TX (US)

(73) Assignee: ALLIANCE PARTNERS LLC, San Antonio, TX (US)

(21) Appl. No.: 16/626,065

(22) PCT Filed: Jun. 22, 2018
(86) PCT No.: PCT/US2018/039095

§ 371 Date: Dec. 23, 2019

Related U.S. Application Data
(60) Provisional application No. 62/523,687, filed on Jun. 22, 2017.

(51) Int. Cl. A61B 17/70; A61B 17/80

(52) U.S. Cl. CPC A61B 17/7059; A61B 17/8042

(57) ABSTRACT

A device and method for locking a bone fastening or fixation mechanism in a receiving member of a medical implant. The bone fastening or fixation mechanism is used to attach the receiving member to a bone structure in the medical implant. The locking mechanism prevents the bone fastening mechanism for backing out. The locking mechanism is a multi-mode locking mechanism. In some embodiments, the locking device has three settings, an automatic mode setting (soft locked position), a manual mode setting (hard locked position), and a neutral mode setting (unlocked position). In some embodiments, the locking device has two settings, a manual mode setting (hard locked position) and a neutral mode setting (unlocked position).

### CROSS-REFERENCE TO RELATED APPLICATIONS

This application claims priority benefits to U.S. Patent Application Ser. No. 62/523,687, filed on Jun. 22, 2017, entitled “Medical Implant Having An Automatic And Manual Locking Mechanism And Method Of Use Thereof,” which patent application is commonly assigned to the Assignee of the present invention and is hereby incorporated herein by reference in its entirety for all purposes FIELD OF INVENTION

A medical implant having a locking mechanism for locking and retaining a bone fastening or fixation mechanism and method of use thereof.

### BACKGROUND OF INVENTION

The spine is the axis of the skeleton on which all of the body parts hang. In humans, the normal spine has seven cervical segments, twelve thoracic segments, five lumbar segments, five sacral segments (which fuse to form the sacrum) and three to five coccygeal segments (which fuse to form the coccyx. The lumbar spine attaches to the pelvis, and in turn is supported by the hip and leg bones. The bony vertebral bodies of the spine are separated by intervertebral discs, which act as joints but allow known degrees of flexion, extension, lateral bending, and axial rotation and translation.

Typical vertebra has a thick anterior bone mass called the vertebral body, with a neural (vertebral) arch that arises from the posterior surface of the vertebral body. The centers of adjacent vertebrae are supported by intervertebral discs. The disc and/or vertebral bodies may be displaced or damaged due to trauma, disease, degenerative defects, or aging over an extended period of time. One result of this displacement or damage to an intervertebral disc or vertebral body may be chronic back pain. In many cases, to alleviate back pain from degenerated or herniated discs, part or all of the disc is removed and may be replaced with an implant that promotes fusion of the remaining bony anatomy.

The success or failure of spinal fusion may depend upon several factors. For instance, the spacer or implant or cage used to fil the space left by the removed disc must be sufficiently strong to support the spine under a wide range of loading conditions. The spacer should also be configured so that it likely to remain in place once it has been positioned in the spine by the surgeon, surgical physician's assistant, etc. Additionally the material used for the spacer should be biocompatible material and should have a configuration that promotes bony ingrowth.

In combination with spacers or cages, a plating system is used to prevent expulsion of the spacer from the spine during the fusion process. These devices, commonly referred to as bone fixation plating systems (i.e., cervical plate), typically include one or more plates and fasteners (typically screws) for aligning and holding vertebrae in a fixed position with respect to one another. Plating systems independent of the spacers have additional complications such as loosening and failure of the hardware. Two common failures are the breakage of the plates, and the backing out of screws into soft tissues of the patient's body.

Cervical cages are used to stabilize the spine during the fusion process. These devices likewise include one or more plates and fasteners (typically screws) for aligning and holding vertebrae in a fixed position with respect to one another. A common failure for corpectomy cages and cervical cages is, like other medical implants, such as plating systems, is the backing out of screws into soft tissues of the patient's body.

The backing out of the screws is typically a result of the screws failure to achieve a sufficient purchase in the bone, although the stripping of the screws has also been known to cause this problem. Common challenges are that medical implants (such as plating systems and cages) require “carpentry” work to match fit aspects of the vertebral bodies.

Thus, it is important that the medical implant is properly held in place by the fastener. To keep the fastener from backing out, a locking (or retention) device is typically utilized. The locking device physically impedes the anterior portion of the screw from rising, which prevents the fastener from rotating (as this would cause the fastener to rise). Once the locking device is in the “locked” position, the fixation mechanism is held in place. Examples of locking mechanisms known in the art are shown in U.S. Pat. No. 8,702,766, issued Apr. 22, 2014 to Mueller and U.S. Pat. No. 8,641,768, issued Feb. 4, 2014, to Duffield et al.

It is further important that the locking device be properly positionable both in the unlocked position (so that the medical practitioner can have access to properly position and implant the fastener) and the locked position (so that the locking device can properly function). U.S. Pat. No. 9,381,093, issued to Jul. 5, 2016, to Morris et al, discloses and teaches a locking device for fixation mechanism of medial implant in which the locking mechanism is kept in place (both in the locked and unlocked positions) using a retaining mechanism. The retaining mechanism provides predetermined locked and unlocked positions that are readily moved to the practitioner and maintained in position once place there.

However, there remains a need for a locking device that is able to work automatically (i.e., one in which the fixation mechanism can be inserted and then the locking device automatically locks to prevent the fixation mechanism from backing out without any interaction by the medical practitioner) and also able to work manually (i.e., one in which the fixation mechanism is set in the locked or unlocked position and will only be changed by interaction by the medical practitioner.

Accordingly, there is a need for an improved locking device for medical implant systems.

### SUMMARY OF INVENTION

The present invention is a medical implant (plate or cage) that is held in place by a fastening/fixation mechanism, that is, typically, a screw. To keep the screw from backing out, a locking device is utilized. The locking device physically impedes the anterior portion of the screw from rising, which prevents the screw from rotating (as this would cause the screw to rise). The locking mechanism is a multi-mode locking mechanism. The locking device can have three settings, an automatic mode setting (soft locked position), a manual mode setting (hard locked position), and a neutral mode setting (unlocked position). Alternatively, the locking device can have two settings, a manual mode setting (hard locked position) and a neutral mode setting (unlocked position).

In general, in one aspect, the invention features a medical implant. The medical implant includes a receiving member body having a first opening through which a first fastener can be received. The medical implant further include a locking mechanism located near the first opening. The locking mechanism includes an expandable fastener cover including a first expandable fastener cover wall. The first expandable fastener cover wall is operable to move between a closed position and an open position relative to the first opening. When the first expandable fastener cover wall is in the closed position, a portion of the first expandable fastener cover wall covers a portion of the first opening so that the first fastener cannot pass through the first opening. When the first expandable fastener cover wall is in the open position, the first expandable fastener cover wall is not covering any portion of the first opening so that the first fastener can pass through the first opening. The locking mechanism further includes a shiftdisc component rotatably adjacent to the first expandable fastener cover wall. The shiftdisc component includes a body having at least one resilient lever arm and at least one edge portion. The shiftdisc component is operable to be moved among a first mode position, a second mode position, and a third mode position. When the shiftdisc component is positioned in the first mode position, the at least one resilient arm is acting as a spring to resiliently push the portion of the first expandable fastener cover wall over the portion of the first opening and can operate in an automatic mode in which, without the use of a tool, the first fastener (I) can be inserted through the first opening thereby moving the first expandable fastener cover wall from the closed position to the open position to permit insertion of the first fastener and (II) thereafter the insertion of the first fastener through the first opening, the first expandable fastener cover wall automatically moves back from the open position to the closed position to automatically lock the first fastener in place. When the shiftdisc component is positioned in the second mode position, the at least one edge portion is pushing the first expandable fastener cover wall outward such that the first expandable fastener cover wall is set in the closed position. When the shiftdisc component is in the third mode position, the first expandable fastener cover wall is set in the open position.

Implementations of the invention can include one or more of the following features:

The medical implant can be a bone fixating plating system.

The bone fixating plating system can include a cervical, thoracic, or lumbar plate.

The medical implant can be a corpectomy cage.

The medical implant can be a cervical, thoracic, or lumbar cage.

The receiving body can have a second fastener opening. The second fastener opening can be operable for allowing a second fastener to be inserted through the second fastener opening for fixing the medical implant to bone. The expandable fastener cover can further include a second expandable fastener cover wall. The second expandable fastener cover wall can be operable to move between a closed position and an open position relative to the second opening. The locking mechanism can further be located near the second fastener opening and can be rotatably adjacent to the second expandable fastener cover wall. When the second expandable fastener cover wall is in the closed position, a portion of the second expandable fastener cover wall can cover a portion of the second opening so that the second fastener cannot pass through the second opening. When the second expandable fastener cover wall is in the open position, the second expandable fastener cover wall can be not covering any portion of the second opening so that the second fastener can pass through the second opening. When the shiftdisc component is positioned in the first mode position, the second fastener (I) can be inserted through the second opening thereby moving the second expandable fastener cover wall from the closed position to the open position to permit insertion of the second fastener and (II) thereafter the insertion of the second fastener through the opening, the second expandable fastener cover wall can automatically move back from the open position to the closed position to automatically locks the second fastener in place.

The first expandable fastener cover wall and the second expandable cover wall can be separate.

The first expandable fastener cover can be a single connected piece in which the first expandable cover wall and the second expandable cover wall are connected directly by one or more further portions of the expandable fastener cover.

The first fastener can include a screw.

The shiftdisc component can include at least two retaining lever arms.

The shiftdisc component can have a hole operable for receiving a rotatable tool. The shiftdisc component can be rotatable in a clockwise direction and a counter-clockwise direction using the rotating tool.

Wherein the medical implant can have an indicator that can show whether the locking mechanism is in the first mode position, the second mode position, or the third mode position.

The receiving member body can include a plurality of fastener openings and a plurality of locking mechanism. Each of the locking mechanisms can be operable for locking fasteners positioned in at most two of the fastener openings.

Each of the locking mechanisms can be operable for locking fasteners positioned in at most one of the fastener openings.

Each of the receiving member body, the expandable fastener cover, and the shiftdisc component can include a material selected from a group consisting of metal alloys, polymers, ceramics, and composites thereof.

The receiving member body can have a thickness of 0.5 mm to 4 mm and a width of 5 mm to 40 mm.

The receiving member body can be selected from a group consisting of a one level plate, a two level plate, a three level plate, a four level plate, and a five level plate.

The first opening through which a first fastener can be received can be operable to receive a fastener selected from a group consisting of (a) fixed-angle screws, (b) variable-angle screws, (c) self-drilling screws, (d) self-tapping screws, and (e) combinations thereof.

The first expandable fastener cover wall can have a spring like design.

The shiftdisc component can be operable to move from the third mode position to the first mode position by rotating in a first direction. The shiftdisc component can be unable to be rotated in the first direction when the shiftdisc component is in the first mode position. The shiftdisc component can be operable to move from the third mode position to the second mode position by rotating in a second direction that is opposite the first direction. The shiftdisc component can be unable to be rotated in the second direction when the shiftdisc component is in the second mode position.

The medical implant can further include the first fastener received in the first opening.

The fastener can include a material selected from a group consisting of metal alloys, polymers, ceramics, and composites thereof.

In general, in another aspect, the invention features a method that includes the step of selecting a medical implant having a receiving body that includes a first fastener opening and a locking mechanism. The locking mechanism includes an expandable fastener cover that includes a first expandable fastener cover wall. The first expandable fastener cover wall is operable to move between a closed position and an open position relative to the first opening. The locking mechanism further includes a shiftdisc component rotatably adjacent to the first expandable fastener cover wall and operable to move among a first mode position, a second mode position, and a third mode position. When the first expandable fastener cover wall is in the closed position, a portion of the first expandable fastener cover wall covers a portion of the first opening so that the first fastener cannot pass through the first opening. When the first expandable fastener cover wall is in the open position, the expandable fastener cover wall is not covering any portion of the first opening so that the first fastener can pass through the first opening. The method further includes the step of setting the shiftdisc component in the first mode position or the third mode position. The method further includes the step of inserting a first fastener into the first opening. When the shiftdisc component is in the first position, the step of inserting the first fastener into the first opening thereby moves the first expandable fastener cover wall from the closed position to the open position to permit insertion of the first fastener through the first fastener opening. When the shiftdisc component is in the third position, the first expandable cover is in the open position. The method further includes the step of securing the first fastener to a bone. When the shiftdisc component is in the first position, after the insertion of the first fastener through the first opening, the first expandable fastener cover wall automatically moves back from the open position to the closed position to automatically lock the first fastener in place. The method further includes the step of setting the shiftdisc component to the first mode position or the second position, in which the first expandable fastener cover is in the closed position.

Implementations of the invention can include one or more of the following features:

The step of setting the shiftdisc component in the first mode position or the third mode position can include setting the shiftdisc component in the first position.

The step of setting the shiftdisc component in the first mode position or the second mode position can include maintaining the shiftdisc component in the first position.

The step of setting the shiftdisc component in the first mode position or the third mode position can include setting the shiftdisc component in the third position.

The step of setting the shiftdisc component to the first mode position or the second mode position can include rotating the shiftdisc component to the second mode position.

The medical implant can be a bone fixating plating system.

The bone fixating plating system can include a cervical, thoracic, or lumbar plate.

The medical implant can be a corpectomy cage.

The medical implant can be a cervical, thoracic, or lumbar cage.

The medical implant can further include a second fastener opening. The expandable fastener cover can further include a second expandable fastener cover wall. The second expandable fastener cover wall can be operable to move between a closed position and an open position relative to the second opening. The shiftdisc component can be rotatably adjacent to the second expandable fastener cover wall. When the second expandable fastener cover is in the closed position, a portion of the second expandable fastener wall cover can cover a portion of the second opening so that the second fastener cannot pass through the second opening. When the second expandable fastener cover wall is in the open position, the second expandable fastener cover wall can be not covering any portion of the second opening so that the second fastener can pass through the second opening. The method can further include inserting a second fastener into the second fastener opening while the shiftdisc component is in the first mode position or the third mode position. When the shiftdisc component is in the first mode position, the step of inserting the second fastener into the second fastener opening can thereby move the second expandable fastener cover wall from the closed position to the open position to permit insertion of the second fastener through the second fastener opening. After the insertion of the second fastener through the second opening, the expandable fastener cover can automatically move back from the open position to the closed position to automatically lock the second fastener in place. The method can further include securing the second fastener to the bone while the shiftdisc component is in the first mode position or the third mode position. The step of setting the shiftdisc component to the first mode position or the second position can set the first expandable fastener cover in the closed position.

The step of inserting the second fastener into the second fastener opening while the shiftdisc component is in the first mode position or the third mode position can include the shiftdisc component is in the first position.

The step of setting the shiftdisc component to the first mode position or the second position can includes maintaining the shiftdisc component in the first position.

The step of inserting the second fastener into the second fastener opening while the shiftdisc component is in the first mode position or the third mode position can include the shiftdisc component is in the third position.

The step of setting the shiftdisc component to the first mode position or the second position can include rotating the shiftdisc component to the second mode position.

The first fastener can include a screw.

The shiftdisc component can include at least two retaining levers.

The step of rotating the shiftdisc component can include inserting a rotatable tool into a hole of the shiftdisc component and rotating in either a clockwise direction or a counter-clockwise direction by at least 15 degrees.

The step of rotating can include utilizing an indicator on the medical implant to determine whether the locking mechanism is in the first mode, the second mode, or the third mode

The receiving member body can include a plurality of fastener openings and plurality of locking mechanisms. The method can include moving at least one of the locking mechanisms in the plurality of locking mechanisms from the first mode position to the second mode position to lock at least one of the fasteners positioned in at least one of the fastener openings.

The method can include moving at least one of the locking mechanisms in the plurality of locking mechanism to lock at least two of the fasteners.

The method can further include rotating the shiftdisc component to the third mode position, wherein the expandable fastener cover is set in the open position. The method can further include un-securing the first fastener from the bone. The method can further include removing the first fastener through the first fastener opening.

The method can be utilized during a fixation procedure of a cervical, thoracic, or lumbar spine at levels C1 through S1.

Each of the receiving member body, the expandable fastener cover, the shiftdisc component, and the first fastener can include a material selected from a group consisting of metal alloys, polymers, ceramics, and composites thereof.

In general, in another aspect, the invention features a medical implant. The medical implant includes a receiving member body having a first opening through which a first fastener can be received. The medical implant further includes locking mechanism located near the first opening. The locking mechanism include an expandable fastener cover that includes a first expandable fastener cover wall. The first expandable fastener cover wall is operable to move between a closed position and an open position relative to the first opening. When the first expandable fastener cover wall is in the closed position, a portion of the first expandable fastener cover wall covers a portion of the first opening so that the first fastener cannot pass through the first opening. When the first expandable fastener cover wall is in the open position, the first expandable fastener cover wall is not covering any portion of the first opening so that the first fastener can pass through the first opening. The locking mechanism further include a shiftdisc component rotatably adjacent to the first expandable fastener cover wall. The shiftdisc component includes a body having at least one edge portion. The shiftdisc component is operable to be moved between a first mode position and a second mode position When the shiftdisc component is positioned in the first mode position, the at least one edge portion is pushing the first expandable fastener cover wall outward such that the first expandable fastener cover wall is set in the closed position. When the shiftdisc component is positioned in the second mode position, the first expandable fastener cover wall is set in the open position.

Implementations of the invention can include one or more of the following features:

The receiving body can have a second fastener opening. The second fastener opening can be operable for allowing a second fastener to be inserted through the second fastener opening for fixing the medical implant to bone. The expandable fastener cover further can include a second expandable fastener cover wall. The second expandable fastener cover wall can be operable to move between a closed position and an open position relative to the second opening. The locking mechanism can be further located near the second fastener opening and is rotatably adjacent to the second expandable fastener cover wall. When the second expandable fastener cover wall is in the closed position, a portion of the second expandable fastener cover wall can cover a portion of the second opening so that the second fastener cannot pass through the second opening. When the second expandable fastener cover wall is in the open position, the second expandable fastener cover wall can be not covering any portion of the second opening so that the second fastener can pass through the second opening.

In general, in another aspect, the invention features a method that includes the step of selecting a medical implant having a receiving body that include a first fastener opening and a locking mechanism. The locking mechanism includes an expandable fastener cover that includes a first expandable fastener cover wall. The first expandable fastener cover wall is operable to move between a closed position and an open position relative to the first opening. The locking mechanism further includes a shiftdisc component rotatably adjacent to the first expandable fastener cover wall and operable to move between a first mode position and a second mode position. The shiftdisc component includes at least one edge portion. When the first expandable fastener cover wall is in the closed position, a portion of the first expandable fastener cover wall covers a portion of the first opening so that the first fastener cannot pass through the first opening. When the first expandable fastener cover wall is in the open position, the expandable fastener cover wall is not covering any portion of the first opening so that the first fastener can pass through the first opening. The method further includes the step of setting the shiftdisc component in the first mode position such that the first expandable cover wall is in the open position. None of the at least one edges of the shiftdisc component are in contact with the first expandable fastener cover wall. The method further includes the step of inserting a first fastener into the first opening. The method further includes the step of setting the shiftdisc component to the second mode position to position at least one of the at least one edges of the shiftdisc component in contact with the first expandable cover wall so that the first expandable fastener cover wall is set in the closed position.

Implementations of the invention can include one or more of the following features:

The medical implant can further include a second fastener opening. The expandable fastener cover can further include a second expandable fastener cover wall. The second expandable fastener cover wall can be operable to move between a closed position and an open position relative to the second opening. The shiftdisc component can be rotatably adjacent to the second expandable fastener cover wall. When the second expandable fastener cover is in the closed position, a portion of the second expandable fastener wall cover can cover a portion of the second opening so that the second fastener cannot pass through the second opening. When the second expandable fastener cover wall is in the open position, the second expandable fastener cover wall can be not covering any portion of the second opening so that the second fastener can pass through the second opening. The method can further include inserting a second fastener into the second fastener opening while the shiftdisc component is in the first mode. In the first mode, none of the at least one edges of the shiftdisc component can be in contact with the second expandable fastener cover wall. The method can further include securing the second fastener to the bone while the shiftdisc component is in the first mode position. The step of setting the shiftdisc component to the second position can position at least one of the at least one edges of the shiftdisc component in contact with the second expandable cover wall so that the second expandable fastener cover wall is set in the closed position.

The foregoing has outlined rather broadly the features and technical advantages of the invention in order that the detailed description of the invention that follows may be better understood. Additional features and advantages of the invention will be described hereinafter that form the subject of the claims of the invention. It should be appreciated by those skilled in the art that the conception and the specific embodiments disclosed may be readily utilized as a basis for modifying or designing other structures for carrying out the same purposes of the invention. It should also be realized by those skilled in the art that such equivalent constructions do not depart from the spirit and scope of the invention as set forth in the appended claims.

It is also to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of the description and should not be regarded as limiting.

### BRIEF DESCRIPTION OF THE DRAWINGS

For a more complete understanding of the present invention, and the advantages thereof, reference is now made to the following descriptions taken in conjunction with the accompanying drawings, in which:

FIG. 1A is an isometric view of an embodiment of the present invention showing a cervical plate with the locking mechanism in the manually locked position.

FIG. 1B is an enlarged view of the locking mechanism shown in embodiment of FIG. 1.

FIG. 2A is the isometric view of the embodiment of FIG. 1A with a portion of the embodiment transparent to show parts of the locking mechanism.

FIG. 2B is a cross-sectional, transverse view of the embodiment of FIG. 1A.

FIGS. 3A-3C are, respectively, anterior, isometric, and sagittal (superior/inferior) views of a bone fastener used with embodiments of the present invention (such as shown in FIG. 1A). (For the features of the medical implant, such as an inserted bone fastener, that is an anterior cervical plate, the anterior view is the front view and the sagittal view is a side view, in the normal orientation of use. Hence, unless otherwise indicated, terms like “anterior,” “posterior,” “sagittal,” “superior,” and “inferior” are describing front, back, side, top, and bottom, respectively for the normal orientation of use of an anterior cervical plate. The “lateral” direction is direction in the plane of the sagittal view, i.e., when sagittal sides move outward or inward relative to one another, these sagittal sides are referred to as moving “laterally” in the normal orientation of use of an anterior cervical plate of the present invention).

FIGS. 4A-4B are, respectively, anterior and posterior isometric views of the expandable fastener cover of the locking mechanism shown in FIG. 2A.

FIGS. SA-5B are, respectively, anterior and posterior isometric views of the shiftdisc component of the locking mechanism shown in FIG. 2A.

FIG. 6 is an anterior view of locking mechanism illustrated in FIG. 2A, with the locking mechanism set in the automatic mode setting (the soft locked position).

FIG. 7 is an anterior view of locking mechanism illustrated in FIG. 2A, with the locking mechanism set in the manual mode setting (the hard locked position).

FIG. 8 is an anterior view of locking mechanism illustrated in FIG. 2A, with the locking mechanism set in the neutral mode setting (the unlocked position).

FIG. 9A is an alternative embodiment of the present invention in which the expandable fastener cover is in multiple parts. The locking mechanism set in the neutral mode setting (the unlocked position).

FIG. 9B is another alternative embodiment of the present invention in which the expandable fastener cover is in multiple parts. The locking mechanism is in the automatic mode setting, and is in the open (unlocked) position.

FIG. 9C is an enlarged view of the locking mechanism shown in embodiment of FIG. 9B.

FIGS. 9D-9E are, respectively, anterior and posterior views of the shiftdisc component of the locking mechanism shown in FIGS. 9B-9C.

FIG. 9F is an anterior of the expandable fastener shown in FIGS. 9B-9C having two separate sagittal walls.

FIG. 10A is an alternative embodiment of the present invention in which there is an indicator showing the set mode of the device.

FIGS. 108-10C are, respectively, anterior and posterior isometric views of the shiftdisc component of the locking mechanism shown in FIG. 1A.

FIG. 11A is an alternative embodiment of the present invention with an alternative expandable fastener cover that is guided.

FIGS. 11B-11C are, respectively, anterior and posterior isometric views of the expandable fastener cover of the locking mechanism shown in FIG. 11A.

FIG. 12A is an isometric view of an embodiment of the present invention showing a cervical plate with an alternative locking mechanism in the manually locked position.

FIG. 12B is an enlarged view of the locking mechanism shown in embodiment of FIG. 12A.

FIG. 13A is anterior view of an embodiment of the present invention showing a linear cervical plate with the locking mechanism in the unlocked position.

FIG. 13B is an enlarged view of the locking mechanism shown in embodiment of FIG. 13A.

FIGS. 14A-14E are various alternative bone fasteners that can be used in embodiments of the present invention.

### DETAILED DESCRIPTION

The technology relates to device and method for locking a bone fastening or fixation mechanism in a receiving member of a medical implant. The bone fastening or fixation mechanism is used to attach the medical implant to a bone structure. The locking mechanism prevents the bone fastening or fixation mechanism for backing out. The locking mechanism is a multi-mode locking mechanism. The locking device can have three settings, an automatic mode setting (soft locked position), a manual mode setting (hard locked position), and a neutral mode setting (unlocked position). Alternatively, it can have two settings, a manual mode setting (hard locked position) and a neutral mode setting (unlocked position).

As discussed in more detail below, when set in the automatic mode setting, the locking device is capable of being moved from the locked position to the unlocked position to allow entry of the fastener and then will automatically return to the locked position. When, however, the manual mode setting or neutral setting is utilized, the locking device is not retained in the locked and unlocked position, respectively. I.e., when in the manual mode setting, the locking device is retained in the locked position, and when in the neutral setting, the locking device is retained in the unlocked position.

The locking mechanism of the present invention in its various embodiments has significant advantageous. In some embodiments, it provides an automatic mode setting, such that each fastener is automatically locked in place without further manipulation by the surgeon after positioning the fastener in place. It also provides a mode for a surgeon to affirmatively and, until the mode is changed, permanently lock the locking mechanism to prevent the fastener from unfastening itself. It also provides a mechanism to quickly unlock the locking mechanism should the need arise to back out the fastener.

Another advantage of the present invention is that it provides hard and quickly identifiable stops when moving among the settings (three mode setting/two mode settings). It also precludes over-rotation by the practitioner when implanting the cervical plate because the expandable fastening cover is not being rotated, but rather its expansion is being controlled by the movement of the shiftdisc component located within the expandable fastening cover. Moreover, once the locking device is set in place, automatic, manual, or neutral, as the case may be, it will be maintained in that position. The locking device maintains its low profile because the shiftdisc component is able to fit in the space within the expandable fastening cover.

While the figures refer to medical implant that is an anterior cervical plate, the multiple phase locking mechanism can be used in other plates and in other types of medical implants, such as cages.

### Three Mode-Setting Locking Device

In some embodiments of the present invention, a three mode-setting locking device is utilized (i.e., a device that has a hard locked position, a soft locked position, and an unlocked position).

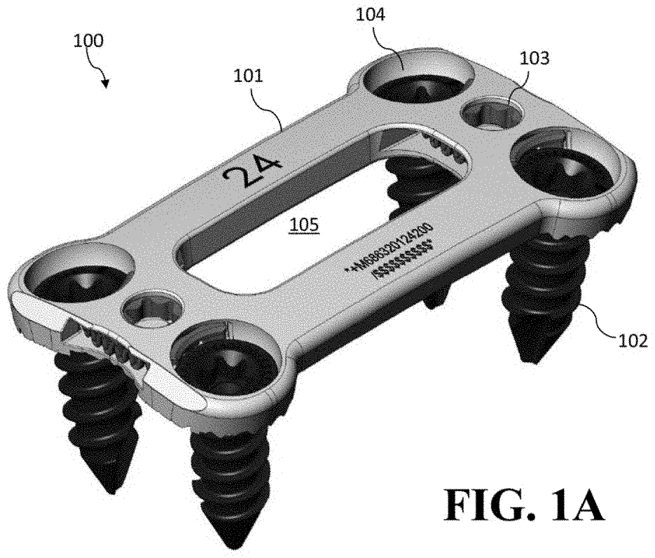

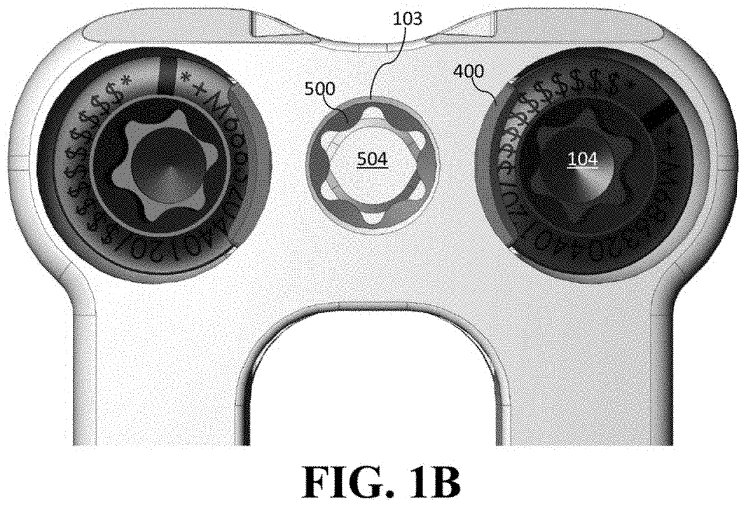

Referring to the figures, FIG. 1A is an isometric view of an anterior cervical plate assembly 100 with a plate portion 101 and a locking mechanism 103. The locking mechanism 103 keeps the bone fasteners 102 from backing out of holes 104. The locking device 103 physically impedes the anterior portion of the fastener 102 (i.e., screw head) from rising, which prevents the fastener 102 from rotating (as this would cause the fastener 102 to rise). When the locking device 103 is in the “locked” (or closed) position, the fastening/fixation mechanism 102 is held in place.

The locking device 103 can operate in manual (“M”), automatic (“A”), and neutral (“N”) modes. The plate portion 101 also has a window 105.

FIG. 1B reflects a magnified view of the locking mechanism 103 shown in FIG. 1A, which can be rotated into one of three modes of configurations (mode settings), namely (a) automatic setting (soft locked position); (b) manual setting (hard locked position); and (c) neutral (unlocked, in which the fastener can be removed). These modes of configurations are available by turning an inner shift disc (such as by around 60 degree increments) between modes. As shown in FIG. 1A (and discussed further below in the following figures), when the locking mechanism 103 is in the automatic setting, a rotation of around 60 degree in the clockwise direction will set the locking mechanism in the manual setting. Typically, in some embodiments this is the farthest that the locking mechanism can be turned in the clockwise direction, so the user (such as a surgeon) cannot over rotate the locking mechanism past the manual setting. As further shown in FIG. 1A (and discussed further below in the following figures), when the locking mechanism 103 is in the automatic setting, a rotation of around 60 degree in the counter-clockwise direction will set the locking mechanism in the neutral setting. Typically, in some embodiments this is the farthest that the locking mechanism can be turned in the counter-clockwise direction, so the user (such as a surgeon) cannot over rotate the locking mechanism past the neutral setting. The mode settings typically can be hard/tactile and can have an audible click between modes. While the description discusses the use of the present invention by a surgeon, other users such as operators, surgical physician's assistant, etc. similarly apply.

The automatic mode setting is one in which the surgeon can insert the fastener into the opening and attach it to the bone, ater which the fastener is locked in place without the surgeon having to operate the locking mechanism. The manual mode is one in which the locking mechanism is set in the locked position and the fastener can neither be inserted or removed without changing the surgeon changing the mode setting. The neutral mode is one in which the expandable fastener cover is entirely disengaged from, and does not interact with, the fastener so that the fastener is readily inserted or removed by the surgeon. However, the fastener cannot be locked in position without the surgeon changing the mode setting from the neutral mode.

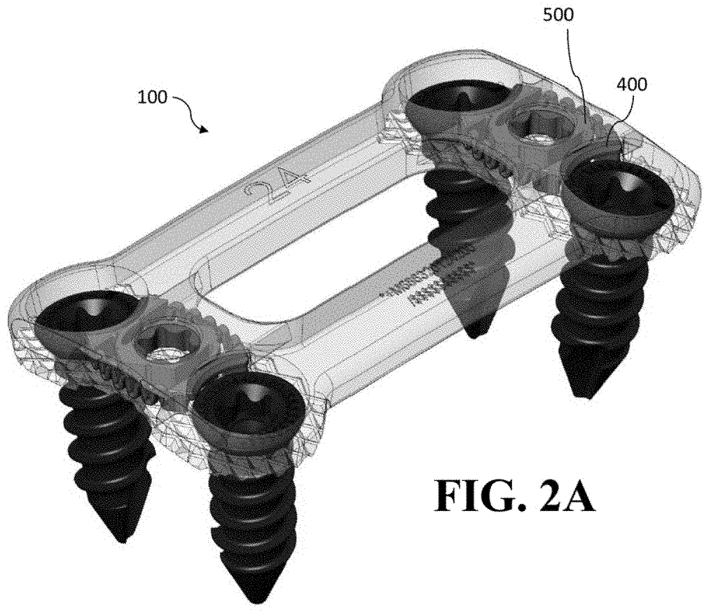

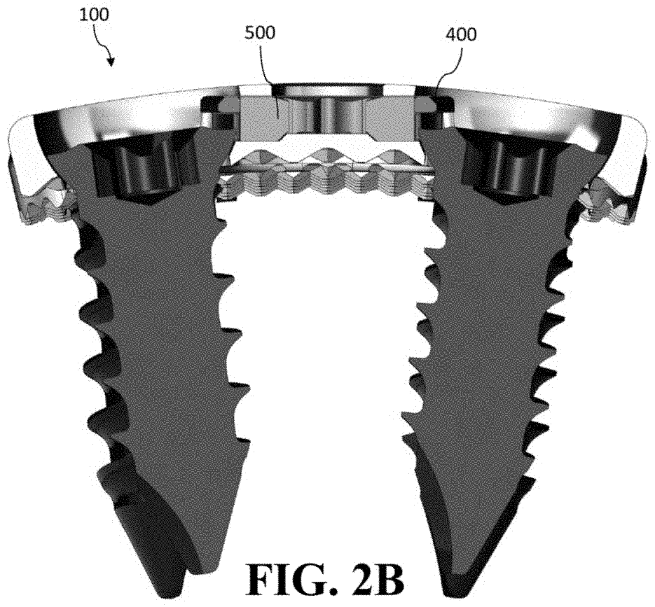

FIG. 2A is the isometric view of anterior cervical plate assembly 100 with plate portion 101 transparent to show parts of the locking mechanism 103, namely expandable fastener cover 400 and shiftdisc component 500. FIG. 2B is cross-sectional, transverse view of anterior cervical plate assembly 100 and likewise shows fastener cover 400 and shiftdisc component 500.

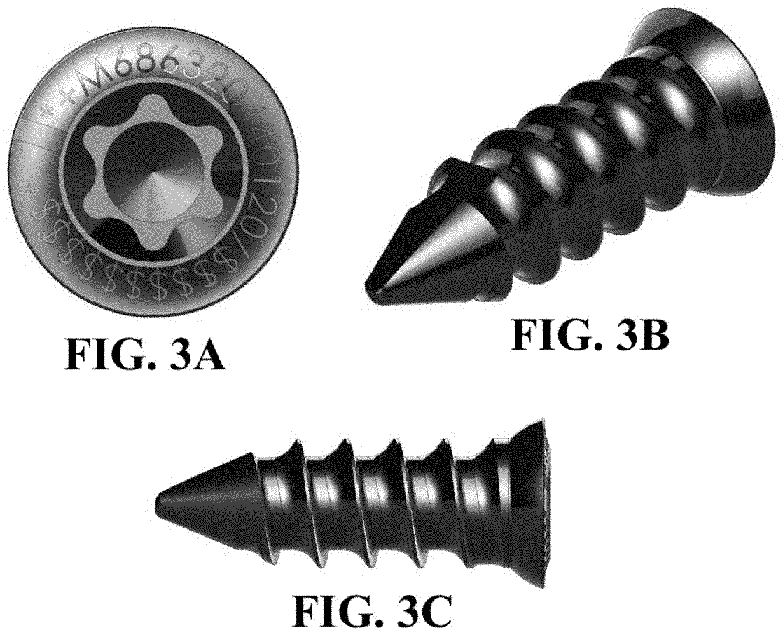

FIGS. 3A-3C are, respectively, anterior, isometric, and sagittal (superior/inferior) views of bone fastener 102 shown in FIG. 1A.

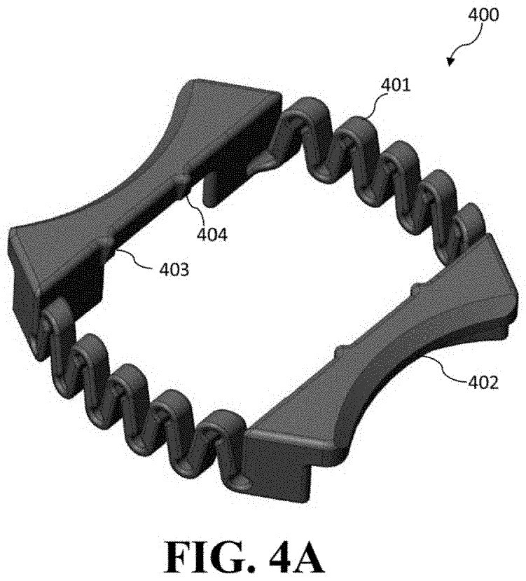

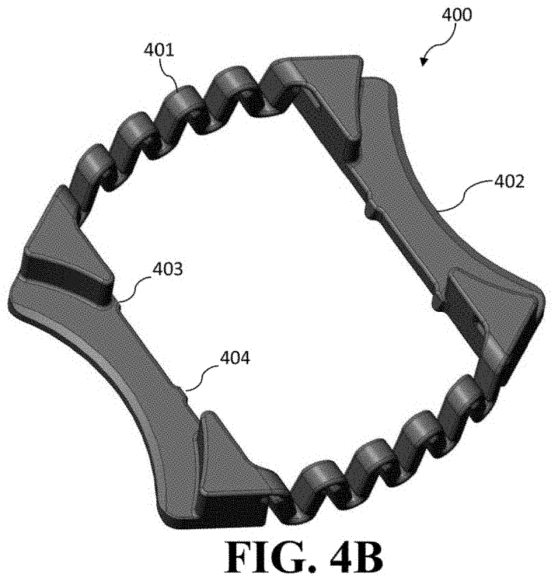

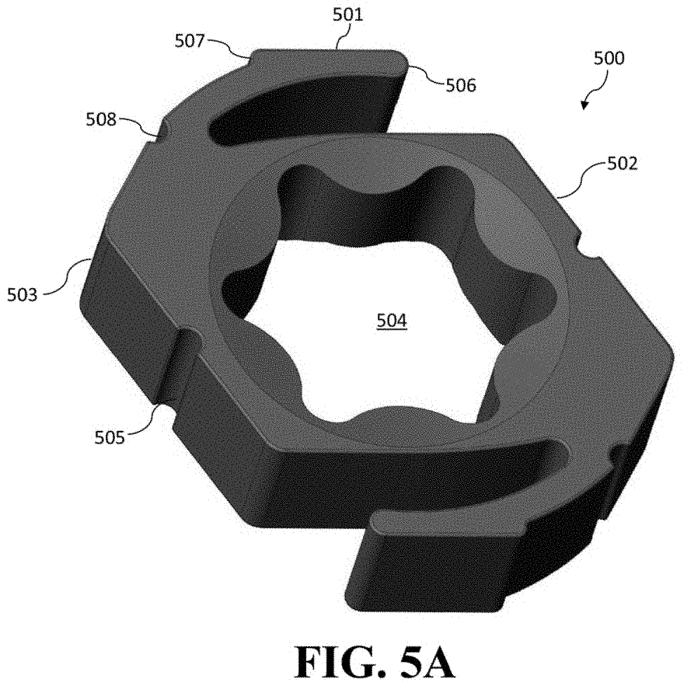

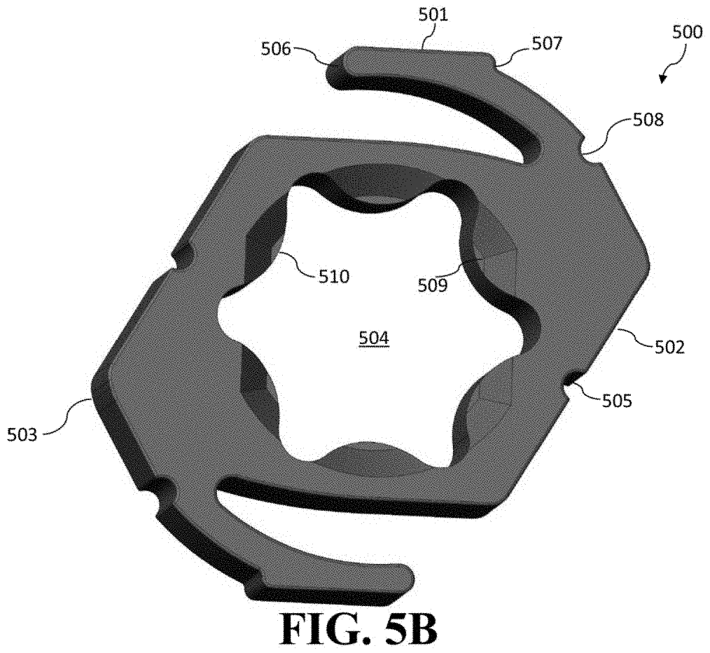

FIGS. 4A-4B are, respectively, anterior and posterior isometric views of expandable fastener cover 400 of the locking mechanism 103. Per the orientation of FIGS. 4A-4B, expandable fastener cover 400 has superior/inferior sides 401 and sagittal sides 402. Per the orientation of anterior cervical plate assembly 100, the superior/inferior sides 401 are superior and inferior, while the sagittal sides 402 are sagittal. It is sagittal sides 402 that are the parts of expandable fastener cover 400 that are expanded across the anterior of fastener 102 to keep the fasteners from backing out. Sagittal sides 402 can include bumps 403 and 404, which assist in the proper positioning of the mode settings, i.e., they provide some tactile and auditory indicators to the surgeon to indicate that the locking mechanism 103 has been properly set in the desired mode setting. As also shown in FIG. 4A-4B, the superior/inferior sides 401 are in a spring like shape so that these are readily expanded upon application of an outward force in a lateral direction). The spring shape then allows the superior/inferior sides 401 to return to their contracted shape after the outward force is no longer applied.

FIGS. SA-5B are, respectively, anterior and posterior isometric views of shiftdisc component 500 of the locking mechanism 103. Shiftdisc component 500 has two resilient lever arms 501, two sides 502, and two edge portions 503 surrounding its outer circumference. The middle of shiftdisc component is hole 504 (such as the illustrated star-shaped hole 504), in which a rotatable tool can be inserted for rotating shiftdisc component 500. For that reason, shiftdisc component is rotatable position in the inferior portion of expandable fastener cover 400. Each of the resilient lever arms 501 has a free end 506 and a hump 507. Indents 505 (located on each of sides 502) and indents 508 (located on or at the base of each resilient lever arm 501 toward the respective edge portion 503) can also be present. It is the indents 505 and 503, free end 506, and hump 507 that can interact with bumps 403 and 404, (when they come into contact) to assist in the proper positioning of the mode settings, such as by providing some tactile and auditory indicators to the surgeon to indicate that the locking mechanism 103 has been properly set in the desired mode setting. These also work together to maintain the shiftdisc component 500 in the selected mode due to friction between one or more of the indents 505 and 508, free end 506, and hump 507 and the appropriate bumps 403 and 404, based upon the selected mode.

The expandable fastener cover 400 is operable to cover a portion of the opening (or pair of openings) to keep the fastener 102 from backing out. The shiftdisc component 500 rotates within the expandable fastener cover 400 so that it can operate in the different mode settings depending upon the particular setting. Indents 509 and 510 are also located on the posterior side of shiftdisc component 500 to control the rotation of the shiftdisc component by interacting with the features (such as hump features on plate portion 101) that indents 509 and 510 come in contact with. By such contacts of intents 509 and 510 with such hump features, this prevents the shiftdisc component 500 from over-rotation by the surgeon.

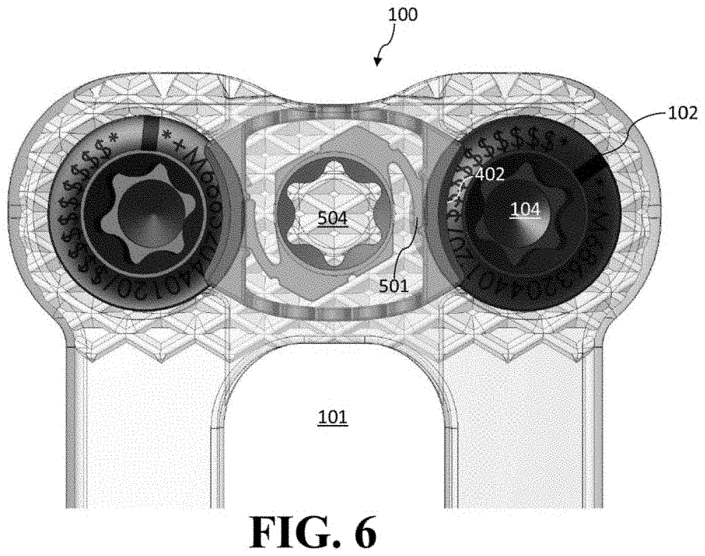

Referring to FIG. 6, this is an anterior view of locking mechanism 103 with the locking mechanism 103 set in the automatic mode setting (soft locked position). Anterior cervical plate assembly 100 includes plate portion 101 having a pair of openings (openings 104). Moving inward (i.e., contracting) is when the sagittal sides 402 of expandable fastener cover 400 of the expandable fastener cover 400 contract laterally toward the middle allowing a fastener 102 to be able to be moved through an opening 104. (Superior/inferior sides 401 contract when this occurs in view of the spring like design of superior/inferior sides 401).

Again, as illustrated in FIG. 6, the locking mechanism 103 is in the automatic mode setting. The free ends 506 and a hump 507 are positioned by bumps 404 and 403, respectively, so that the lever arms 501 are properly positioned on sagittal sides 402, which holds the shiftdisc component 500 in the automatic mode setting. In this automatic mode setting, the lever arms 501 are thus in position to act as a spring, pushing sagittal sides 402 of the expandable fastener cover 400 outward laterally.

In this automatic mode setting (soft locked position), the surgeon can insert the fastener 102, such as a tapered screw shown in FIGS. 3A-3C, into opening 104. As the head feature of the fastener passes through opening 104, this causes sagittal side 402 to be pushed inward allowing the head feature of the fastener 102 to go completely through opening 104. Once the head feature of the fastener 102 passes through, the inward pushing on sagittal side 402 is no longer occurring, and lever arms 501 will push outward laterally to return the expandable fastener cover 400 back into the closed position as shown in FIG. 6 with the sagittal sides 402 partially covering openings 104. This automatically prevents the fastener 102 from backing out. Thus, the expandable fastener cover 400 is being pushed outward by the lever arms 501 to expand sagittal sides 402 outward when in automatic mode.

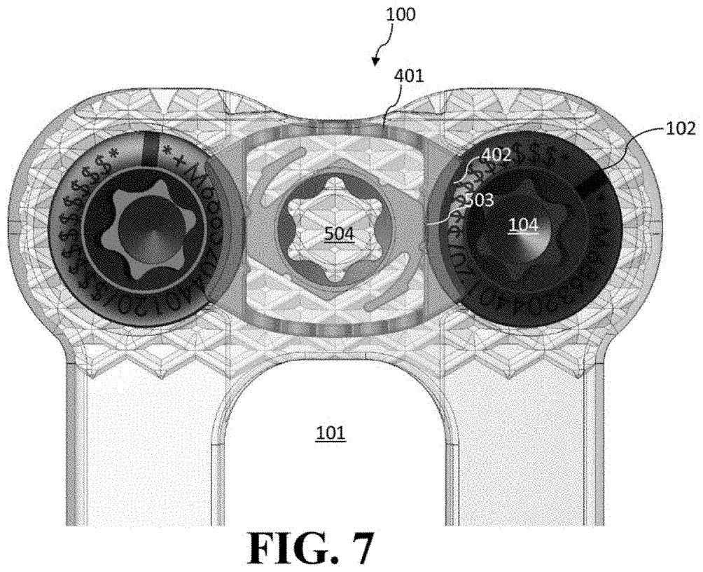

Referring to FIG. 7, this this is an anterior view of locking mechanism 103 with the locking mechanism 103 set in the manual mode setting (hard locked position).

In this manual mode setting, the shiftdisc component 500 has been rotated to the manual mode setting. I.e., in FIG. 7, the shiftdisc component 500 is rotated around 60 degrees clockwise from the automatic mode setting, which is the position of the shiftdisc component 500 shown in FIG. 6. When in this position, indent 508 is in contact with bump 404, which maintains the shiftdisc component 500 in the manual mode setting. This engagement of the indent 508 and bump 404 further provides the surgeon with tactile and audible indicators that the surgeon has set the shiftdisc component 500 in the manual mode setting. Again, such rotation in a clockwise direction past the manual mode setting may be prevented by one of the indents 509 and 510 located on the posterior side of shiftdisc component 500.

By such rotation, the edge portions 503 of the shiftdisc component 500 have now pushed sagittal sides 402 outward so that expandable fastener cover 400 is now covering opening 104 and preventing the fastener 102 from being able to move through. Since edge portion 503 is not resilient (like resilient lever arms 501), the sagittal sides 402 will not move inward (to open the openings 104) even if a surgeon attempts to insert a fastener 102 through opening 104. Hence, in this manual mode setting, a fastener 102 can neither be inserted or removed because the expandable fastener cover 400 is then in the closed (and hard locked) position.

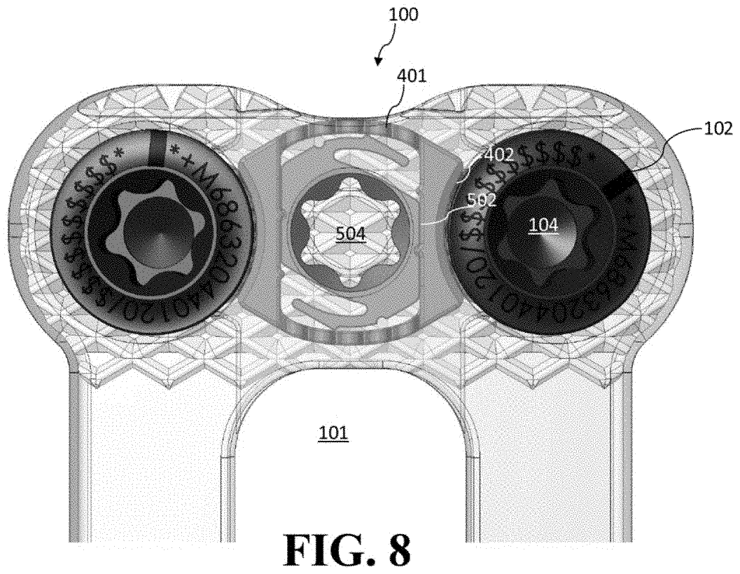

Referring to FIG. 8, this this is an anterior view of locking mechanism 103 with the locking mechanism 103 set in the neutral mode setting (unlocked position).

In this neutral mode setting, the shiftdisc component 500 has been rotated to the neutral mode setting. I.e., in FIG. 8, the shiftdisc component 500 is rotated around 60 degrees counter-clockwise from the automatic setting, which is the position the shiftdisc component 500 shown in FIG. 6 and a total of around 120 degrees counter-clockwise as compared with the position of the shiftdisc component 500 shown in FIG. 7. When in this position, indent 505 is in contact with bump 404, which maintains the shiftdisc component 500 in the neutral mode setting. This engagement of the indent 505 and bump 404 further provides the surgeon with tactile and audible indicators that the surgeon has set the shiftdisc component 500 in the neutral mode setting. Again, such rotation in a counter-clockwise direction past the neutral mode setting may be prevented by one of the indents 509 and 510 located on the posterior side of shiftdisc component 500.

By such rotation of the shiftdisc component 500 into the neutral mode setting, the sides 502 of the shiftdisc component 500 are facing sagittal sides 402 of expandable fastener cover 400 and are not pushing sagittal sides 402 outward (so that sagittal sides 402 will naturally be positioned inward due to spring like shape of superior/inferior sides 401) and not covering any portion of openings 104. (When there are no forces are being applied to expandable fastener cover 400, its shape would be as shown in FIG. 8). Such position of sagittal sides 402 thus allows the fastener 102 to be able to move through opening 104 in either direction. Since sides 502 are not pushing to provide an outward force (like lever arms 501), the sagittal sides 402 of expandable fastener cover 400 will not move outward (to close the openings) to prevent a fastener 102 from being inserted or removed. In such setting, the expandable fastener cover 400 is then in the unlocked (or open) position.

In one use of anterior cervical plate (or device)100, anterior cervical plate (or device)100 is used in the automatic mode setting. The surgeon is able to insert a fastener 102 into the opening 104, which will then automatically lock fastener 102 in place.

Optionally, once implanted, the surgeon can manually lock the locking mechanism 103 by using the rotatable tool to rotating the shiftdisc component 500 into the manual mode setting for the locking mechanism 103.

If desired to remove the fastener 102, the surgeon can thereafter move the locking mechanism 103 into the neutral mode setting (which, for example, opens opening 104 for fastener 102 removal). In such instance, the surgeon can then reverse out the fastener 104.

In another use of anterior cervical plate (or device) 100, the surgeon can move the locking mechanism 103 to the neutral mode setting before inserting the fastener 102 (as this opens opening 104). Once, the fastener 102 is in place, the surgeon can then change the settings (typically to the manual mode setting) to lock the fasteners 102 in place (i.e., the expandable fastener cover 400 is then placed in the hard closed position). If desired, the fastener 102 can then be removed similar as described above in which the surgeon reverses out the fastener 102 from opening 104 when the mode of the locking mechanism 103 is set in the neutral mode setting.

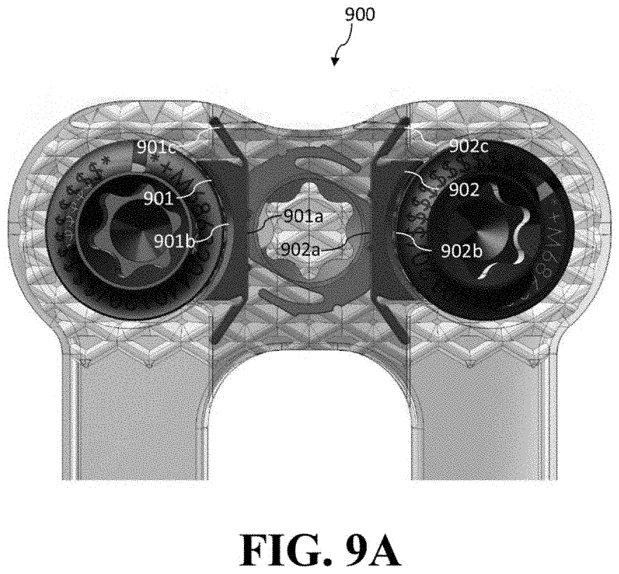

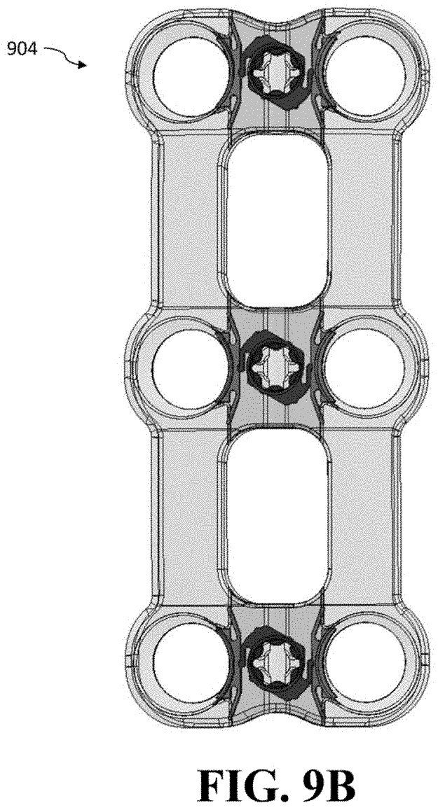

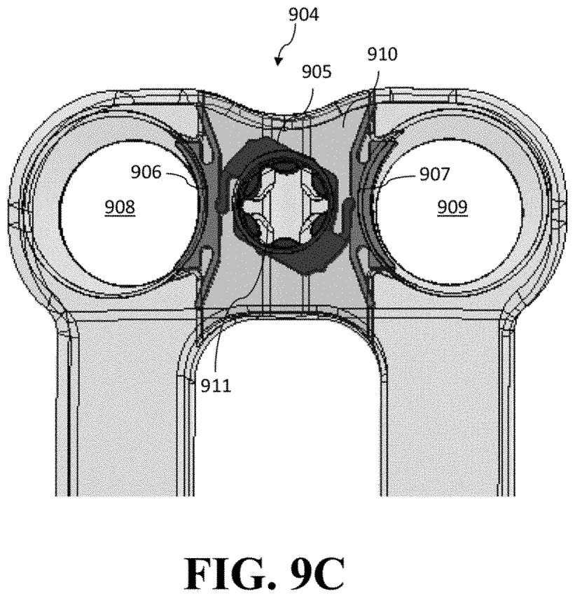

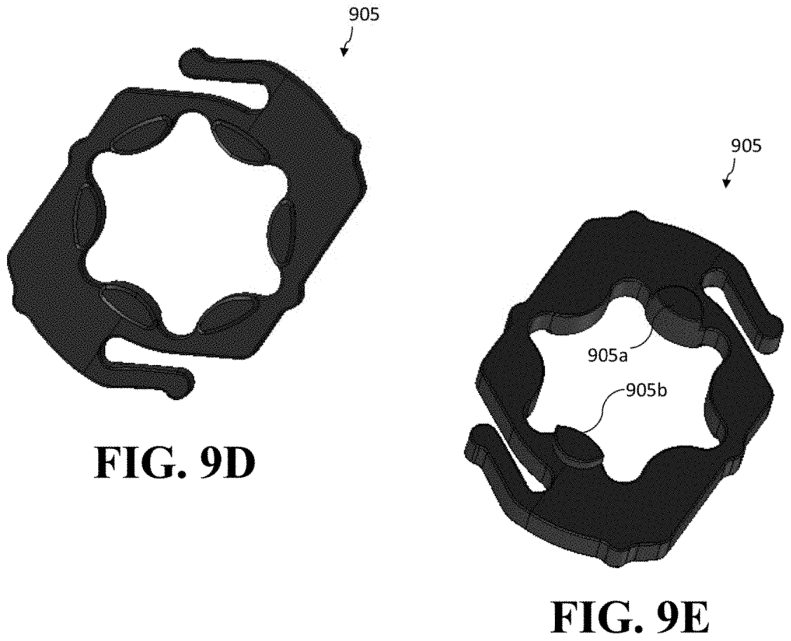

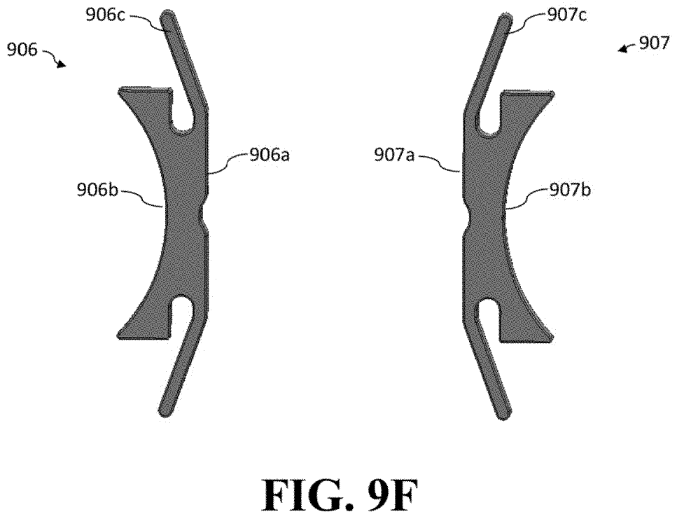

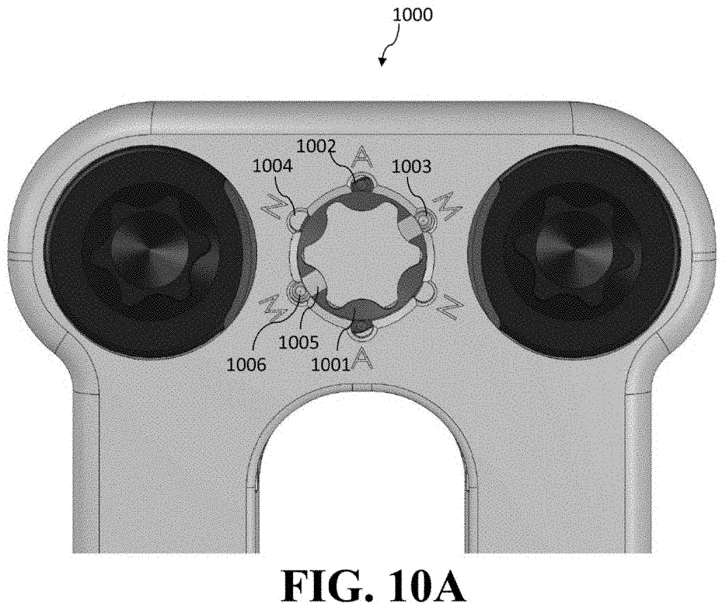

FIG. 9A illustrates anterior cervical plate (or device)900 in which the expandable fastener 400 has been replaced with an expandable fastener that is multiple parts (sagittal walls 901 and 902). Sagittal walls 901 and 902 function similarly as sagittal walls 402, but without superior/inferior walls 401. Each of sagittal walls 901 and 902 have a generally flat side (sides 901a and 902a, respectively) that contact the locking mechanism. Each of sagittal walls 901 and 902 also have a generally crescent side (sides 901b and 902b, respectively) that are the portions of sagittal walls 901 and 902 that would cover a portion of the respective openings of anterior cervical plate 900 when in the locked position. Each of sagittal walls 901 and 902 also has wings (wings 901c and 902c, respectively) that provide an inward force to sagittal walls 901 and 902, to compensate for the absence superior/inferior walls 401 and the forces that arise due to the present of superior/inferior walls 401 in expandable fastener cover 400 shown in FIG. 4, and discussed above. Anterior cervical plate (or device)900 would operate the same as described above.

FIGS. 9B-9C illustrate anterior cervical plate (or device) 904, which like the anterior cervical plate 900 shown in FIG. 9A, has an expandable fastener that is multiple parts (sagittal walls 906-907). FIGS. 9D-9E are, respectively, anterior and posterior views of the shiftdisc component 905 of anterior cervical plate 904. FIG. 9F is an anterior of the expandable fastener shown of the locking mechanism anterior cervical plate (or device) 904, which has two separate sagittal walls 906 and 907.

The orientation of shiftdisc component 905 is different than that shown in anterior cervical plate assembly 100 shown in FIGS. 6-8 (and other figures) and discussed above. As shown in FIGS. 9B-9C, shiftdisc component 905 is set so that the locking mechanism is in the automatic mode setting. The locking mechanism is in the automatic mode setting, and is in the open (unlocked) position, i.e., while not shown in FIGS. 9B-9C, fasteners are partially inserted into holes 908 and 909 such that forces are applied outward (laterally) to position the sagittal walls 906-907 in the opened position.

As shown in FIGS. 9-9C, the free ends of the lever arms of shiftdisc component 905 are properly positioned on generally flats sides 901a and 902a of, respectively, sagittal walls 906-907, which holds the shiftdisc component 905 in the automatic mode setting. Similar to anterior cervical plate assembly 100 as shown in FIG. 6, in this automatic mode setting, the lever arms of shiftdisc component 905 are thus in position to act as a spring, pushing upon generally flats sides 901a and 902a of sagittal walls 906-907 outward laterally. Each of sagittal walls 906 and 907 also have a generally crescent side (sides 906b and 906b, respectively) that are the portions of sagittal walls 906 and 907 that would cover a portion of the respective openings of anterior cervical plate 904 when in the locked position. Each of sagittal walls 906 and 907 also has wings (wings 906c and 906c, respectively) that provide an inward force to sagittal walls 906 and 907. Anterior cervical plate (or device)904 would operate the same as described above.

Plate 910 of anterior cervical plate 904 has arc slot notches 911 (shown in FIGS. 9B-9C) that interact with the hardstop portions 905a and 905b located on the posterior side of shiftdisc component 905 (shown in FIG. 9E). This controls the rotation of the shiftdisc component 905 relative to plate 910. For the embodiment shown in FIGS. 9B-9C, in the automatic mode setting shown in FIG. 9B-9C, the hardstop portions 905a and 905b prevent the shiftdisc component from rotating in the counterclockwise direction, and permit rotation 120 degrees in a clockwise direction.

Per the orientation of FIGS. 98-9C, the shiftdisc component 905 can be rotated to the neutral mode setting (unlocked position) by rotating the shiftdisc component 905 around 60 degrees clockwise from the automatic mode setting.

In this neutral mode setting (unlocked position), indents and bumps can maintains the shiftdisc component 905 in the neutral mode setting. This engagement of indents and bumps further provides the surgeon with tactile and audible indicators that the surgeon has set the shiftdisc component 905 in the neutral mode setting (unlocked position).

By such rotation of the shiftdisc component 905 into the neutral mode setting (unlocked position), the portion of shiftdisc component 905 facing the generally flat sides 906a-907a of expandable sagittal walls 906-907, respectively, are not pushing the expandable sagittal walls 906-907 outward laterally (so that the expandable sagittal walls 906-907 will be naturally be positioned inward due to spring like shape of the wings 906c-907c, respectively, of expandable sagittal walls 906-907 and not covering any portion of openings 908-909, respectively (i.e., no portions of crescent sides 906b-906b are covering openings 908-909). Such position of the expandable sagittal walls 906-907 (and specifically crescent sides 906b-906b) thus allows fasteners to be able to move through each of openings 908-909 in either direction. Since, in this neutral mode setting (unlocked position) the sides of shiftdisc component 905 in contact with the generally flat sides 906a-907a of the expandable sagittal walls 906-907 are not pushing to provide an outward force (like the lever arms of shiftdisc component 905), the crescent sides 906b-906b of the expandable sagittal walls 906-907 will not move outward (to close the openings) to prevent fasteners from being inserted or removed. In such setting, the expandable fastener cover (made of expandable sagittal walls 906-907) is then in the unlocked (or open) position.

Per the orientation of FIGS. 9B-9C, the shiftdisc component 905 can be rotated to the manual mode setting (hard locked position) by rotating the shiftdisc component 905 around 120 degrees clockwise from the automatic mode setting (which is 60 degrees of clockwise rotation past the neutral mode setting).

When in this hard locked position, indents and bumps can be utilized to maintain the shiftdisc component 905 in the manual mode setting (hard locked position). Again, this engagement further provides the surgeon with tactile and audible indicators that the surgeon has set the shiftdisc component 905 in the manual mode setting. Notches 911 (shown in FIGS. 9B-9C) interact with the hardstop portions 905a and 905b located on the posterior side of shiftdisc component 905 (shown in FIG. 9E to prevent further rotation past 120 degrees in a clockwise direction. Thus, a rotation completely as possible in the clockwise direction will set the locking mechanism 905 in the manual mode setting, while a rotation completely as possible in the counter-clockwise direction will set the locking mechanism 905 in the automatic mode setting (such as shown in FIGS. 9B-9C). The intermediate setting between the two is the neutral mode setting. Accordingly, by rotating the locking mechanism in either a complete clockwise or counterclockwise direction (so that it reaches a hardstop), the locking mechanism is in a locked position (either hard locked or soft locked, as the case may be).

By such rotation to the manual mode setting, the edge portions of the shiftdisc component 905 now will push the generally flat sides 906a-907a of sagittal walls 906-907 outward so that a portion of crescent walls 906b-907b are now covering openings 908-909, respectively and will prevent the fasteners from being able to move through such openings. Since the edge portions of shiftdisc component 905 are not resilient (like the resilient lever arms), the sagittal walls 906-907 will not move laterally inward (so the generally flat sides 906a-907a cannot move to open the openings 908-909, respectively) even if a surgeon attempts to insert a fastener through any of openings 908-909. Hence, similar to anterior cervical plate assembly 100 as shown in FIG. 7, in this manual mode setting, a fastener can neither be inserted or removed because the expandable sagittal walls 906-907 of the expandable fastener are then in the closed (and hard locked) position.

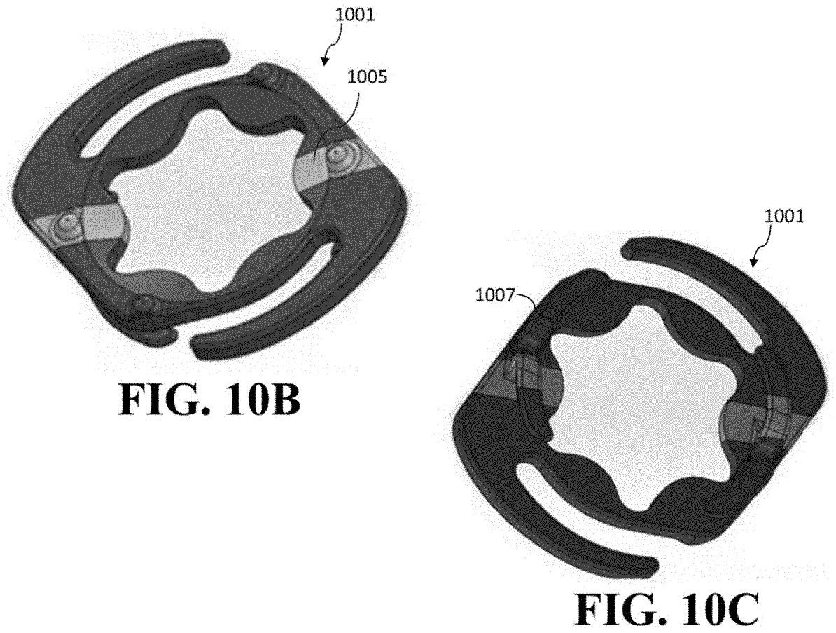

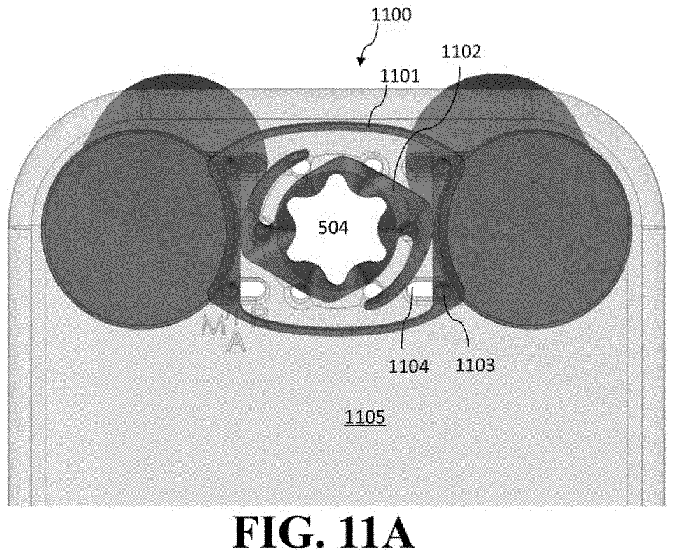

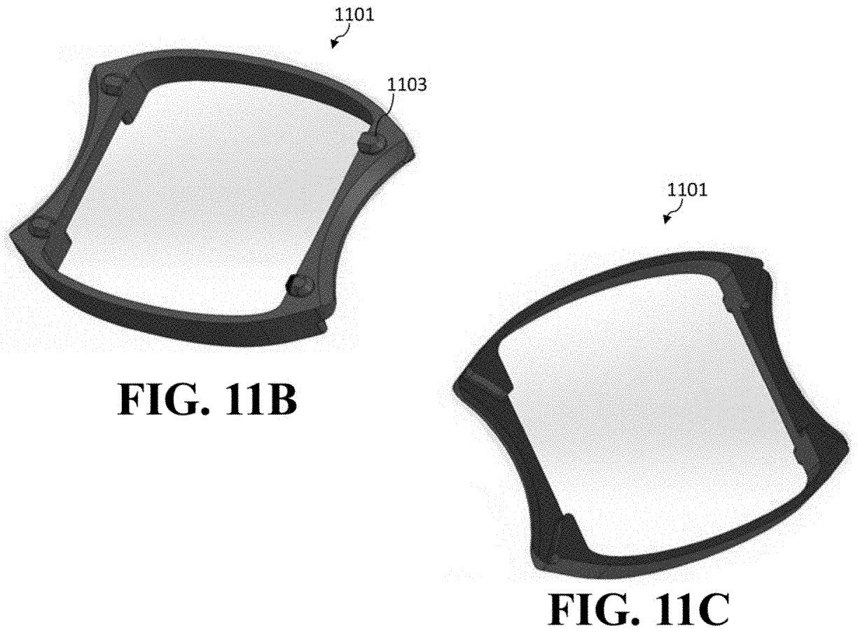

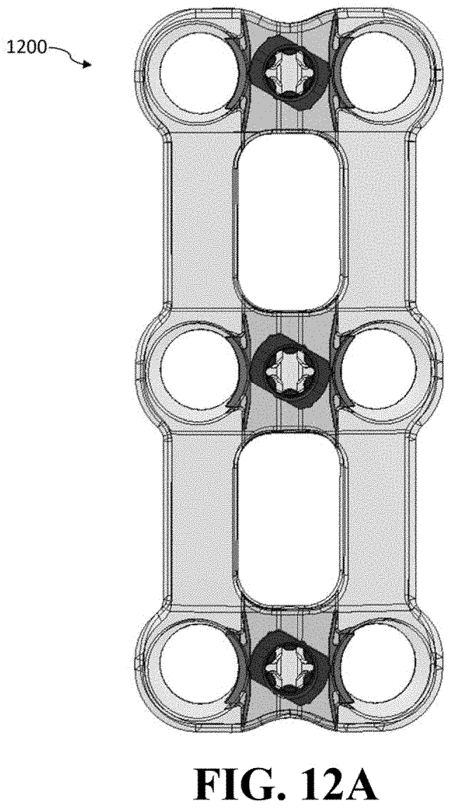

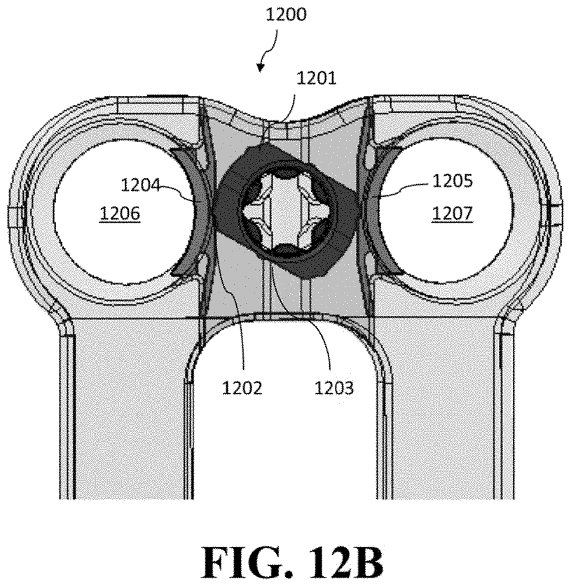

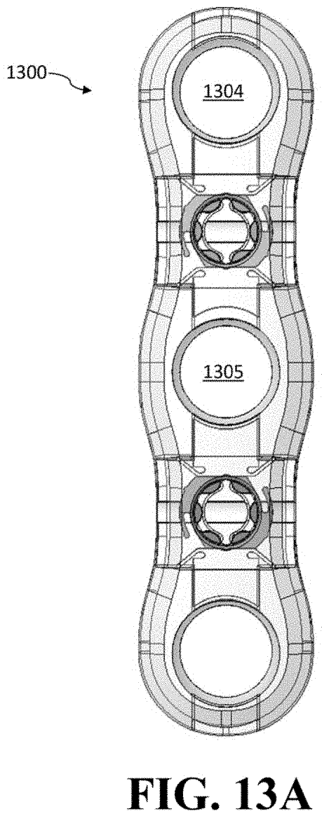

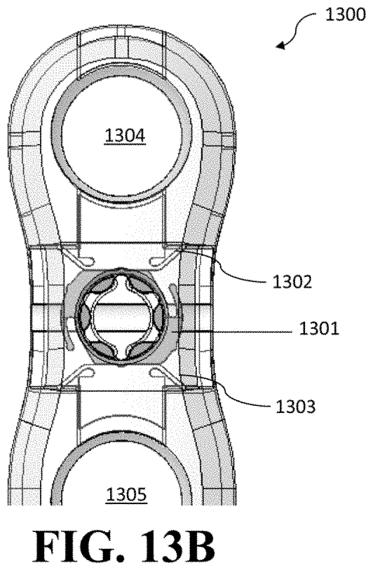

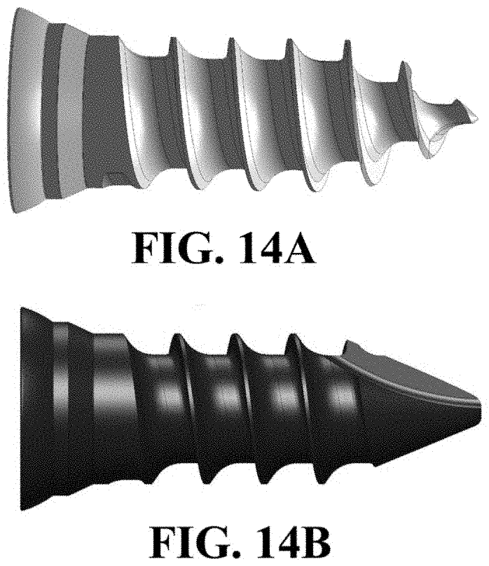

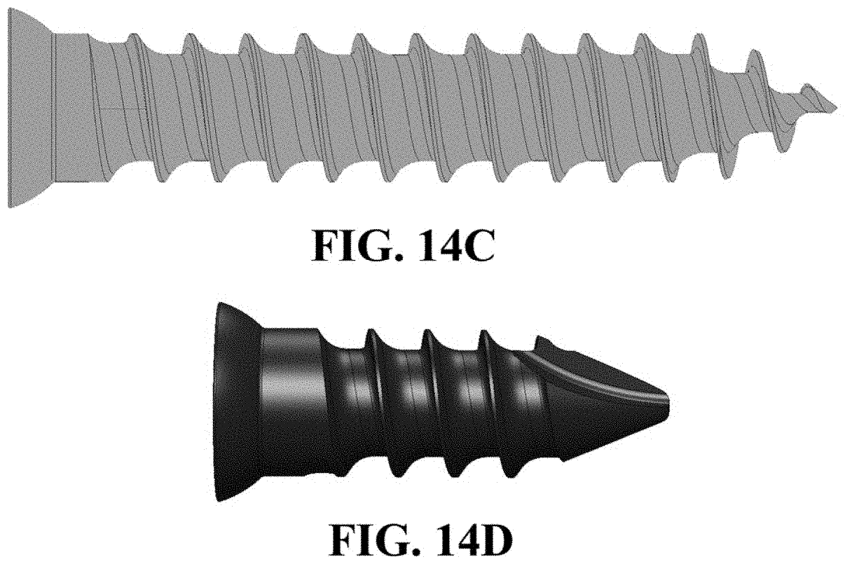

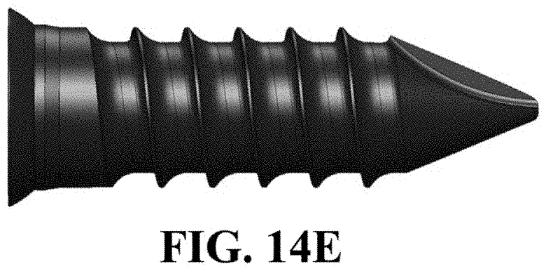

FIG. 10A illustrates anterior cervical plate (or device) 1000 that has an indicator that shows the show the various automatic, manual, and neutral mode settings of anterior cervical plate (or device) 1000 as the locking mechanism is rotated. The locking mechanism again has modes of configurations (mode settings), namely (a) automatic setting (soft locked); (b) manual setting (hard locked); and (c) neutral (unlocked). These modes of configurations are available by turning an inner shift disc (such as by around 60 degree increments) between modes. The mode settings can again be hard/tactile and can have an audible click between modes. The setting is shown in the medical implant by the indication of “A” (for automatic setting 1002), “M” (for manual setting 1003), and “N” (for neutral setting 1004).

The setting is shown in the medical implant by the indication of “M” (for manual setting 201), “A” (for automatic setting 202), and “N” (for neutral setting 203).

For convenience of the surgeon, there may be a colored strip 1005 that indicates which of modes of configurations to which the shiftdisc component 1001 is then set. As illustrated in FIG. 10A, a yellow stripe 1005 indicates the shiftdisc component 1001 is set in the manual mode setting 1003.

FIGS. 10B-10C are, respectively, anterior and posterior views of the shiftdisc component 1001. The yellow strip portion 1005 of shiftdisc component 1001 coincides with the indicators shown in FIG. 10A (i.e., indication of “A” for automatic setting 1002, “M” for manual setting 1003, and “N” for neutral setting 1004). Pin portions 1006 can also be also positioned on shiftdisc component 1001 so that these will snap into place within the indicators shown in FIG. 10A. This provides for some further tactile and auditory indicators to the surgeon to indicate that the locking mechanism has been properly set in the desired mode setting. Portions 1007 may also be located on the posterior side of shiftdisc component 1001 to assist in the proper rotation and mode setting and can also exert some outward spring forces in some settings.

It should be noted that this indicator with colored strip 1005 on anterior cervical plate (or device) 1000 provides the surgeon further visual information as to what mode the anterior cervical plate (or device) 1000 is set in. Hence, the hard stops to prevent over-rotating clockwise (past the manual mode setting) and to prevent over-rotating counter-clockwise (past the neutral mode setting) are typically removed from this embodiment. In such embodiments, if the shiftdisc component 1001 was in the neutral setting 1004 and then rotated an additional 60 degrees counter-clockwise, it has now been rotated into the position the manual setting 1003. Therefore, the rotatable tool can be used to rotate the shiftdisc component 1001 either clockwise or counter-clockwise to reach the desired mode setting.

FIG. 11A illustrates anterior cervical plate (or device) 1100 in which the expandable fastener 400 has been replaced with a guided expandable fastener 1101. While expandable fastener is shown with superior/inferior walls, such superior/inferior walls do not have the spring-like shape similar to superior/inferior walls 401. While such spring-like shape can be utilized in this embodiment, expandable fastener 1101 reflects a different design in which the superior/inferior walls will bow inward and outward depending upon the applied lateral forces applied, such as by shiftdisc component 1102. As shown in FIG. 11A shiftdisc component is in the manual setting, and thus is locked in place providing an outward lateral force.

Expandable fastener 1101 is guided in that it has pin portions 1103 that control the movement outward and inward of the expandable fastener cover 1101 as it expands and contracts. Plate portion 1105 also includes slot channels 1104 in which pin portions 1103 can slide along outward and inward as expandable fastener cover 1101 expands and contracts. Moving outward (i.e., expanding) is when the sagittal sides of expandable fastener cover 1101 move outward laterally and cover a portion of the opening. (Superior/inferior sides of expandable fastener cover 1101 coordinately stretch and move inward when this occurs). Slot channels 1104 control the motion of the expandable fastener cover 1101.

### Two Mode-Setting Locking Device

In some embodiments of the present invention, a two mode-setting locking device is utilized (i.e., a device that has a hard locked position and an unlocked position). While this device has a manual mode and a neutral mode, it does not have an automatic mode (soft locked position).

FIGS. 12A-12B illustrate anterior cervical plate (or device) 1200, which like the anterior cervical plates 900 and 904 shown in FIGS. 9A-9C, has an expandable fastener that is multiple parts (sagittal walls 1204-1205, which are similar to sagittal walls 906 and 907 discussed above and shown in FIG. 9F.). The shiftdisc component 1201 is different than that shown in anterior cervical plate assemblies 100, 900, and 904 shown in FIGS. 6-8 and FIGS. 9A-9C (and other figures) and discussed above. As shown in FIGS. 12A-12B, shiftdisc component 1201 is set so that the locking mechanism is in the manual mode setting (hard locked position). That that edge portions 1202 are in contact with the generally flat sides of sagittal walls 1204-1205.

When in this hard locked position, indents and bumps can be utilized to maintain the shiftdisc component 1201 in the manual mode setting (hard locked position). As before, this engagement further provides the surgeon with tactile and audible indicators that the surgeon has set the shiftdisc component 1201 in the manual mode setting.

In this position, the edge portions 1202 of the shiftdisc component 1201 now will push the generally flat sides of sagittal walls 1204-1205 outward so that portions of crescent sides of sagittal walls 1204-1205 are now covering openings 1206-1207, respectively, and will prevent the fasteners from being able to move through such openings. Since the edge portions 1202 of shiftdisc component 1201 are not resilient, the sagittal walls 1204-1205 will not move laterally inward (to open the openings 1206-1207, respectively) even if a surgeon attempts to insert a fastener through any of openings 1206-1207. Hence, similar to anterior cervical plate assembly 100 as shown in FIG. 7, in this manual mode setting, a fastener can neither be inserted or removed because the expandable sagittal walls 1204-1205 of the expandable fastener are then in the closed (and hard locked) position.

Per the orientation of FIGS. 12A-12B, the shiftdisc component 1201 can be rotated to the neutral mode setting (unlocked position) by rotating the shiftdisc component 905 around 60 degrees clockwise from the manual mode setting (hard locked position). It can be further rotated around another 60 degrees clockwise and be again set in a manual mode setting or rotated around 60 degrees counter-clockwise to return to the manual mode setting shown in FIGS. 12A-12B. I.e., once the shiftdisc component is in the neutral position, it can rotated 60 degrees clockwise or counter-clockwise to set the locking mechanism in either of two manual mode settings (hard locked). There are hard stops that would prevent further rotation of the shiftdisc component past this 120 degrees of rotation, such as described above, so whether rotated completely clockwise or counter-clockwise, the locking mechanism is set in the manual mode setting (hard locked position).

When in this neutral mode setting (unlocked position), indents and bumps can maintains the shiftdisc component 1201 in the neutral mode setting. This engagement of indents and bumps further provides the surgeon with tactile and audible indicators that the surgeon has set the shiftdisc component 1201 in the neutral mode setting (unlocked position).

By such rotation of the shiftdisc component 1201 into the neutral mode setting (unlocked position), the sides 1203 of shiftdisc component 1201 face the generally flat sides of the expandable sagittal walls 1204-1205 and are not pushing the expandable sagittal walls 1204-1205 outward laterally so that the crescent sides of the expandable sagittal walls 1204-1204 will naturally be positioned inward (due to spring like shape of the expandable sagittal walls 1204-1205) and no portions of the crescent sides the expandable sagittal walls 1204-1204 will be covering any portion of openings 1206-1207, respectively.

Such position of the expandable sagittal walls 1204-1204 thus allows fasteners to be able to move through each of openings 1206-1207 in either direction. Since, in this neutral mode setting (unlocked position) the sides 1203 of shiftdisc component 1201 in contact with the expandable sagittal walls 1204-1205 are not pushing to provide an outward force, the expandable sagittal walls 1204-1205 of the expandable fastener cover will not move outward (to close the openings) to prevent fasteners from being inserted or removed. In such setting, the expandable fastener cover (made of expandable sagittal walls 1204-905) is then in the unlocked (or open) position.

### Plates Variations

While the figures refer to medical implant that is an anterior cervical plate, the multiple phase locking mechanism can be used in other plates and in other types of medical implants, such as cages.

This system can be utilized for anterior screw fastening/fixation to the cervical spine and can include a variety of shapes and sizes of plates (bone plates) and fasteners (screws). For example, in addition to as shown above, the locking mechanism can be used a linear cervical plate, such as shown in FIGS. 13A-13B. FIGS. 13A-13B show a linear cervical plate 1300 with the three-mode locking mechanism 1301 in the neutral mode setting (unlocked position). The locking mechanism 1301 is similar to locking mechanism 905 of anterior cervical plate (or device) 904. The superior wall 1302 and the inferior wall 1303 are similar to the sagittal walls 906-907 of anterior cervical plate (or device) 904, and the interaction between locking mechanism 1301 and superior wall 1302 and inferior wall 1303 is similar to as described above for anterior cervical plate (or device) 904. By rotation of the locking mechanism 1301, this will provide the surgeon the ability to pass fasteners through openings 1304 and 1305. It should be noted that for linear cervical plate 1300, opening 1305 has two locking mechanisms. To pass a fastener into the opening 1305, this requires the surgeon to position both of the locking mechanisms adjacent to opening 1305 into modes in which a fastener can be inserted (both neutral mode, both automatic mode, or one neutral mode and the other automatic mode). To remove a fastener through opening 1305, this requires the surgeon to position both of the locking mechanisms adjacent to opening 1305 into the neutral mode. Similar, a fastener is locked in place in opening 1305, as long as one of the adjacent locking mechanism is set in either the automatic mode or the manual mode.

### Components and Fasteners

The components can be manufactured from any implant grade metal alloy, such as titanium alloy (Ti 6Al 4V ELI as described by ASTM F136), as well as ceramic, polymer, or composites thereof. In some embodiments of the present invention, the sterile components are provided. In other embodiments of the present invention, the non-sterile components are provided, which will then need to be steam sterilized by a hospital or surgeon prior to use.

Additional features and benefits of the present invention include:

Typical thickness and width of the plate is 0.5-4.0 mm×15-40 mm.

The length of the plate can include one, two, three, four, and five level plates. (Plate, such as cervical plates, are generally referred to by the number of levels that they overlie, wherein the word “level” refers to the number of intervening intervertebral spaces that are spanned. Thus, for example, a three level cervical plate would span the four vertebrae beyond and between the three intervertebral spaces).

Serrated, textures, and/or knurled under surfaces for grip during fastener placement/delivery.

Large graft window allows for optimal visualization during placement.

Fasteners can be in fixed or variable angle and/or self-drilling/self-tapping, which allow the surgeon to create a variety of constructs. FIG. 14A illustrates a fixed-angle, self-drilling fastener. FIG. 14B illustrates a fixed-angle, self-tapping fastener. FIG. 14C illustrates a variable-angle, self-drilling fastener. FIG. 14D illustrates a variable-angle, self-tapping fastener. While the size can be varied, FIGS. 14A-14D are ø4.0 to ø4.5 mm fasteners (shank diameter), which can be utilized in anterior cervical plate assemblies 100, 900, and 904 shown in FIGS. 6-8 and FIGS. 9A-9C. FIG. 14E illustrates a larger fastener ø5.5 mm fastener (shank diameter), which can be utilized in linear cervical plate 1300 of FIGS. 13A-13B, which has larger openings 1304-1305 compatible for such larger fasteners. Diameters of the fasteners (shank diameters) can be any size, such as between ø3.5-6.5 mm.

Embodiments of the invention can be used for anterior intervertebral screw fastening/fixation of the cervical spine at levels C2-T1. Embodiments can be used in the temporary stabilization of the anterior spine during the development of cervical spinal fusion in patients with the following indications, including, but not limited to:
Degenerative Disc Disease (as defined by neck pain of discogenic origin with degeneration of the disc confirmed by patient history and radiographic studies)
Trauma (including fractures)
Tumors
Deformities or curvatures (including kyphosis, lordosis, or scoliosis)
Pseudoarthrosis
Failed previous fusions
Spondylolisthesis
Spinal stenosis
Revision surgery

The disclosures of all patents, patent applications, and publications cited herein are hereby incorporated herein by reference in their entirety, to the extent that they provide exemplary, procedural, or other details supplementary to those set forth herein. It will be understood that certain of the above-described structures, functions, and operations of the above-described embodiments are not necessary to practice the present invention and are included in the description simply for completeness of an exemplary embodiment or embodiments. In addition, it will be understood that specific structures, functions, and operations set forth in the above-described referenced patents and publications can be practiced in conjunction with the present invention, but they are not essential to its practice. It is therefore to be understood that the invention may be practiced otherwise than as specifically described without actually departing from the spirit and scope of the present invention.

While embodiments of the invention have been shown and described, modifications thereof can be made by one skilled in the art without departing from the spirit and teachings of the invention. The embodiments described and the examples provided herein are exemplary only, and are not intended to be limiting. Many variations and modifications of the invention disclosed herein are possible and are within the scope of the invention. Accordingly, the scope of protection is not limited by the description set out above.

1. A medical implant comprising
(a) a receiving member body having a first opening through which a first fastener can be received; and
(b) a locking mechanism located near the first opening, wherein the locking mechanism comprises
(i) an expandable fastener cover comprising a first expandable fastener cover wall, wherein the first expandable fastener cover wall is operable to move between a closed position and an open position relative to the first opening, wherein
(A) when the first expandable fastener cover wall is in the closed position, a portion of the first expandable fastener cover wall covers a portion of the first opening so that the first fastener cannot pass through the first opening, and
(B) when the first expandable fastener cover wall is in the open position, the first expandable fastener cover wall is not covering any portion of the first opening so that the first fastener can pass through the first opening, and
(ii) a shiftdisc component rotatably adjacent to the first expandable fastener cover wall, wherein the shiftdisc component comprises a body having at least one resilient lever arm and at least one edge portion, wherein
(A) the shiftdisc component is operable to be moved among a first mode position, a second mode position, and a third mode position,
(B) when the shiftdisc component is positioned in the first mode position, the at least one resilient arm is acting as a spring to resiliently push the portion of the first expandable fastener cover wall over the portion of the first opening and can operate in an automatic mode in which, without the use of a tool, the first fastener (I) can be inserted through the first opening thereby moving the first expandable fastener cover wall from the closed position to the open position to permit insertion of the first fastener and (II) thereafter the insertion of the first fastener through the first opening, the first expandable fastener cover wall automatically moves back from the open position to the closed position to automatically lock the first fastener in place,
(C) when the shiftdisc component is positioned in the second mode position, the at least one edge portion is pushing the first expandable fastener cover wall outward such that the first expandable fastener cover wall is set in the closed position, and
(D) when the shiftdisc component is in the third mode position, the first expandable fastener cover wall is set in the open position.

2. The medical implant of claim 1, wherein the medical implant is a bone fixating plating system.

3. The medical implant of claim 2, wherein the bone fixating plating system comprises a cervical, thoracic, or lumbar plate.

4. The medical implant of claim 1, wherein the medical implant is a corpectomy cage.

5. The medical implant of claim 1, wherein the medical implant is a cervical, thoracic, or lumbar cage.

6. The medical implant of claim 1, wherein
(a) the receiving body has a second fastener opening,
(b) the second fastener opening is operable for allowing a second fastener to be inserted through the second fastener opening for fixing the medical implant to bone,
(c) the expandable fastener cover further comprises a second expandable fastener cover wall, wherein the second expandable fastener cover wall is operable to move between a closed position and an open position relative to the second opening,
(d) the locking mechanism is further located near the second fastener opening and is rotatably adjacent to the second expandable fastener cover wall,
(e) when the second expandable fastener cover wall is in the closed position, a portion of the second expandable fastener cover wall covers a portion of the second opening so that the second fastener cannot pass through the second opening,
(f) when the second expandable fastener cover wall is in the open position, the second expandable fastener cover wall is not covering any portion of the second opening so that the second fastener can pass through the second opening, and
(g) when the shiftdisc component is positioned in the first mode position, the second fastener (I) can be inserted through the second opening thereby moving the second expandable fastener cover wall from the closed position to the open position to permit insertion of the second fastener and (II) thereafter the insertion of the second fastener through the opening, the second expandable fastener cover wall automatically moves back from the open position to the closed position to automatically locks the second fastener in place.

7. The medical implant of claim 6, wherein the first expandable fastener cover wall and the second expandable cover wall are separate.

8. The medical implant of claim 6, wherein the first expandable fastener cover is a single connected piece in which the first expandable cover wall and the second expandable cover wall are connected directly by one or more further portions of the expandable fastener cover.

9. The medical implant of claim 1, wherein the first fastener comprises a screw.

10. The medical implant of claim 1, wherein the shiftdisc component comprises at least two retaining lever arms.

11. The medical implant of claim 1, wherein the shiftdisc component has a hole operable for receiving a rotatable tool, wherein the shiftdisc component is rotatable in a clockwise direction and a counter-clockwise direction using the rotating tool.

12. The medial implant of claim 1, wherein the medical implant has an indicator that can show whether the locking mechanism is in the first mode position, the second mode position, or the third mode position.

13. The medical implant of claim 1, wherein
(a) the receiving member body comprises a plurality of fastener openings and a plurality of locking mechanisms, and
(b) each of the locking mechanisms is operable for locking fasteners positioned in at most two of the fastener openings.

14. The medical implant of claim 13, wherein each of the locking mechanisms is operable for locking fasteners positioned in at most one of the fastener openings.

15. The medical implant of claim 1, wherein each of the receiving member body, the expandable fastener cover, and the shiftdisc component comprises a material selected from a group consisting of metal alloys, polymers, ceramics, and composites thereof.

16. The medical implant of claim 1, wherein the receiving member body has a thickness of 0.5 mm to 4 mm and a width of 5 mm to 40 mm.

17. The medical implant of claim 1, wherein the receiving member body is selected from a group consisting of a one level plate, a two level plate, a three level plate, a four level plate, and a five level plate.

18. The medical implant of claim 1, wherein the first opening through which a first fastener can be received is operable to receive a fastener selected from a group consisting of (a) fixed-angle screws, (b) variable-angle screws, (c) self-drilling screws, (d) self-tapping screws, and (e) combinations thereof.

19. The medical implant of claim 1, wherein the first expandable fastener cover wall has a spring like design.

20. The medical implant of claim 1, wherein
(a) the shiftdisc component is operable to move from the third mode position to the first mode position by rotating in a first direction,
(b) the shiftdisc component is unable to be rotated in the first direction when the shiftdisc component is in the first mode position,
(c) the shiftdisc component is operable to move from the third mode position to the second mode position by rotating in a second direction that is opposite the first direction, and
(d) the shiftdisc component is unable to be rotated in the second direction when the shiftdisc component is in the second mode position.

21. The medical implant of claim 1 further comprising the first fastener received in the first opening.

22. The medical implant 21, wherein the fastener comprises a material selected from a group consisting of metal alloys, polymers, ceramics, and composites thereof.

23. A method comprising the steps of:
(a) selecting a medical implant having a receiving body that comprises a first fastener opening and a locking mechanism, wherein
(i) the locking mechanism comprises
(A) an expandable fastener cover that comprises a first expandable fastener cover wall, wherein the first expandable fastener cover wall is operable to move between a closed position and an open position relative to the first opening, and
(B) a shiftdisc component rotatably adjacent to the first expandable fastener cover wall and operable to move among a first mode position, a second mode position, and a third mode position,
(ii) when the first expandable fastener cover wall is in the closed position, a portion of the first expandable fastener cover wall covers a portion of the first opening so that the first fastener cannot pass through the first opening, and
(iii) when the first expandable fastener cover wall is in the open position, the expandable fastener cover wall is not covering any portion of the first opening so that the first fastener can pass through the first opening,
(b) setting the shiftdisc component in the first mode position or the third mode position;
(c) inserting a first fastener into the first opening, wherein
(i) when the shiftdisc component is in the first position, the step of inserting the first fastener into the first opening thereby moves the first expandable fastener cover wall from the closed position to the open position to permit insertion of the first fastener through the first fastener opening, and
(ii) when the shiftdisc component is in the third position, the first expandable cover is in the open position;
(d) securing the first fastener to a bone, wherein, when the shiftdisc component is in the first position, after the insertion of the first fastener through the first opening, the first expandable fastener cover wall automatically moves back from the open position to the closed position to automatically lock the first fastener in place; and
(e) setting the shiftdisc component to the first mode position or the second position, wherein the first expandable fastener cover is in the closed position.

24-45. (canceled)

46. A medical implant comprising
(a) a receiving member body having a first opening through which a first fastener can be received; and
(b) a locking mechanism located near the first opening, wherein the locking mechanism comprises
(i) an expandable fastener cover comprising a first expandable fastener cover wall, wherein the first expandable fastener cover wall is operable to move between a closed position and an open position relative to the first opening, wherein
(A) when the first expandable fastener cover wall is in the closed position, a portion of the first expandable fastener cover wall covers a portion of the first opening so that the first fastener cannot pass through the first opening, and
(B) when the first expandable fastener cover wall is in the open position, the first expandable fastener cover wall is not covering any portion of the first opening so that the first fastener can pass through the first opening, and
(ii) a shiftdisc component rotatably adjacent to the first expandable fastener cover wall, wherein the shiftdisc component comprises a body having at least one edge portion, wherein
(A) the shiftdisc component is operable to be moved between a first mode position and a second mode position,
(B) when the shiftdisc component is positioned in the first mode position, the at least one edge portion is pushing the first expandable fastener cover wall outward such that the first expandable fastener cover wall is set in the closed position,
(C) when the shiftdisc component is positioned in the second mode position, the first expandable fastener cover wall is set in the open position.

47. The medical implant of claim 46, wherein
(a) the receiving body has a second fastener opening,
(b) the second fastener opening is operable for allowing a second fastener to be inserted through the second fastener opening for fixing the medical implant to bone,
(c) the expandable fastener cover further comprises a second expandable fastener cover wall, wherein the second expandable fastener cover wall is operable to move between a closed position and an open position relative to the second opening,
(d) the locking mechanism is further located near the second fastener opening and is rotatably adjacent to the second expandable fastener cover wall,
(e) when the second expandable fastener cover wall is in the closed position, a portion of the second expandable fastener cover wall covers a portion of the second opening so that the second fastener cannot pass through the second opening, and
(f) when the second expandable fastener cover wall is in the open position, the second expandable fastener cover wall is not covering any portion of the second opening so that the second fastener can pass through the second opening.

48-49. (canceled)
